(19) United States
(12) Patent Application Publication
(10) Pub. No.: US 2025/0215451 A1
(43) Pub. Date: Jul. 3, 2025

(54) VERO CELL LINES STABLY EXPRESSING HSV ICP0 PROTEIN

(71) Applicant: THE BRIGHAM AND WOMEN'S HOSPITAL, INC., Boston, MA (US)

(72) Inventor: Feng YAO, Southborough, MA (US)

(73) Assignee: THE BRIGHAM AND WOMEN'S HOSPITAL, INC., Boston, MA (US)

(21) Appl. No.: 18/827,380

(22) Filed: Sep. 6, 2024

Related U.S. Application Data
(62) Division of application No. 16/619,416, filed on Dec. 4, 2019, now Pat. No. 12,098,380.
(60) Provisional application No. 62/515,260, filed on Jun. 5, 2017.

(51) Int. Cl. C12N 15/86; A61K 39/245; C07K 14/005; C12N 5/071; C12N 5/16; C12N 7/02

(52) U.S. Cl. CPC C12N 15/86; A61K 39/245; C07K 14/005; C12N 5/0686; C12N 5/16; C12N 7/025; C12N 2710/16622; C12N 2710/16643; C12N 2710/16652; C12N 2830/003; C12N 2830/60

(57) ABSTRACT

Provided herein are Vero cell lines that stably express Herpes Simplex Virus (HSV) ICP0 protein. These cells have the same morphology of Vero cells, exhibit stable expression of HSV ICP0 protein, and also efficiently complement replication of HSV ICP0 deficient virus for greater than 20, 30, or even 40 cell passages.

### CROSS-REFERENCE TO RELATED APPLICATIONS

This application is a Divisional Application of co-pending U.S. application Ser. No. 16/619,416 filed Dec. 4, 2019, which is a 35 U.S.C. § 371 National Phase Entry Application of International Application No. PCT/US2018/035977 filed Jun. 5, 2018, which designates the U.S. and claims benefit under 35 U.S.C. § 119 (e) of U.S. Provisional Application No. 62/515,260 filed Jun. 5, 2017, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Apr. 19, 2018, is named 043214-086910-WOPT_SL.txt and is 30,637 bytes in size.

### FIELD OF THE INVENTION

Embodiments of the invention are directed to Vero cell lines that stably express Herpes Simplex Virus (HSV) ICP0 protein and to the generation of these cell lines. These cells have the same morphology of Vero cells, exhibit stable expression of HSV ICP0 protein, and also efficiently complement replication of HSV ICP0 deficient virus for greater than 20, 30, or even 40 cell passages.

### BACKGROUND OF THE INVENTION

HSV ICP0 is a multi-functional protein that is required for efficient viral replication at a low multiplicity of infection of HSV in cell cultures (Stow and Stow, J Gen Virol 67:2571-2585, 1986; Sacks and Schaffer, J Virol 61:829-839, 1987; Yao and Schaffer, J Virol 69:6249-6258, 1995). It is a potent activator capable of enhancing gene expression in a promoter independent manner (Hagglund and Roizman, J Virol. 78:2169-2178, 2004). ICP0 interacts with a variety of cellular proteins and can target multiple cellular factors for proteasome-mediated degradation (Hagglund and Roizman J Virol 78:2169-2178, 2004; Liang et al., PNAS 102:5838-5843, 2005; Boutell et al., J Virol 79:12342-12354, 2005; Lilley et al., EMBO J 29:943-955, 2010; Perusina Lanfranca et al., Cell 3:438-454, 2014; Conwell et al., J Virol 89:220-229, 2015). In vivo, deletion of ICP0 significantly reduces the virus ability to establish latent infection as well as the reactivation of virus from latent infection (David A. Leib et al., J Virol 63:759-768, 1989, Cai et al., J Virol 67:7501-7512, 1993; Halford and Schaffer, J Virol 75:3240-3249, 2001; Augustinova H et al., J Virol 78:5756-5765, 2004). ICP0 plays a fundamental role in counteracting host innate antiviral response to HSV infection. It prevents an IFN-induced nuclear block to viral transcription, down regulates TLR2/TLR9-induced inflammatory cytokine response to viral infection, suppresses TNF-α mediated activation of NF-κB signaling pathway, and interferes with DNA damage response to viral infection (reviewed in Perusina Lanfranca et al., Cell 3:438-454, 2014). Moreover, ICP0 is required for efficient translation of vial mRNA in quiescent cells (Walsh and Mohr, Genes & Dev 18:660-672, 2004).

Using the T-REx™ gene switch technology (Invitrogen Inc., CA) (Yao et al., Hum Gene Ther 9:1939-1950) and the dominant-negative mutant polypeptide UL9-C535C of HSV-1 origin of viral replication binding protein UL9, we constructed a novel class of replication-defective HSV-1 recombinants capable of blocking wild-type HSV-1 and HSV-2 infections (dominant-negative) (Yao and Eriksson, Hum Gene Ther 10:1811-1818, 1999; Antiviral Res 53:127-133, 2002; Lu et al. J Invest Dermatol 129:1174-1184, 2009). These non-replicating and dominant-negative HSV-1 recombinant viruses encode 2 copies of UL9-C535C under control of the tetO-bearing HCMV major immediate-early promoter in the HSV-1 ICP0 locus and are replication-competent only in tetracycline repressor-expressing human osteosarcoma cell line U2CEP4R-11, a stable cell line derived from human osteosarcoma U2OS cells. U2OS is a cell line that specifies an activity that can functionally substitute for ICP0 (Yao and Schaffer, 1995, supra) via an unknown mechanism, but in any case such cells are not preferred to manufacture materials for pharmaceutical purposes.

CJ2-gD2 is an HSV-2 ICP0 deletion mutant based non-replicating dominant-negative HSV-2 recombinant virus that we recently constructed, which encodes 2 copies of the gD2 gene at the HSV-2 ICP0 locus driven by the tetO-bearing HSV-1 major immediate-early ICP4 promoter, while gene encoding UL9-C535C is under the control of the tetO-containing hCMV major immediate-early promoter in an opposite orientation of the inserted gD2 gene. While CJ2-D2 expresses little gD2 in tetR-expressing cells, it expresses gD2 as efficiently as wild-type HSV-2 infection in non tetR-expressing cells. CJ2-gD2 is avirulent and incapable of establishing detectable latent infection following immunization. We have demonstrated that CJ2-gD2 can function as an effective vaccine in protecting mice and guinea pigs against wild-type HSV-2 genital infection and disease (Akhrameyeva et al. J Virol 85:5036-5047, 2011; Zhang et al, PLOS ONE 9: e101373; U.S. Pat. No. 8,809,047).

Due to the lack of ICP0 and high-level expression of UL9-C535C, CJ2-gD2 is replication-defective in Vero cells (Akhrameyeva et al. J Virol 85:5036-5047, 2011). Vero cell lines that can complement ICP0 deficient HSV have been generated, however the cell lines have exhibited non-parental cell morphology, slow growth, and loss of complementation efficiency with as little as 15 passages, properties that are not amenable to large scale rigorously regulated clinical production. Sacks and Schaffer describe a Vero cell line that stably expresses HSV-1 ICP0 with an HSV-1 DNA fragment encoding ICP0 gene under its own promoter (Sacks and Schaffer, J Virol 61: 1987). These cells, named 0-28, efficiently complement the growth of HSV-1 ICP0 deletion mutants (Sacks and Schaffer, J Virol 61:829-839, 1987; Cai and Schaffer, J Virol 65:4078-4090, 1991; Yao and Schaffer, J Virol 69:6249-6258, 1995). However, the morphology of 0-28 cells was quite different from the parental Vero cells and the growth of 0-28 cells was significantly slower than the parental Vero cells (Yao and Schaffer, unpublished observation), indicating that expression of ICP0 could be toxic to the cells. Indeed, several studies have indicated cytotoxic effect of ICP0 and its inhibitory effect on cell cycle progression and cell proliferation (Samaniego et al., J Virol 71:4614-4625, 1997; Everett et al., EMBO J 18:1526-1538, 1999; Hobbs et al. J Virol 73:8245-8255, 1999; Lomonte and Everett, J Virol 73:9456-9467, 1999; Guchet D et al., J Gen Med 7:1187-1199, 2005). Samaniego et al. established two ICP0-expressing Vero cell lines, named FO6 and L7, using an ICP0-expressing plasmid similar to that used for 0-28 cells, where ICP0 is expressed using its own ICP0 promoter. FO6 quickly lost its complement efficiency by passage 15. The stability of L7 in complementing growth was not tested and the limiting quantity of ICP0 provided by L7 cells is purported to explain why there is low levels of tk mRNA upon infection with virus (Samaniego et al., J Virol, 1997). Similarly, with ICP0-expressing plasmid, pSH that encodes ICP0 under the ICP0 promoter (Cai and Schaffer, 1989, Yao and Schaffer, 1995), we failed to generate a stable ICP0-expressing Vero cell line that can complement HSV-1 ICP0 deletion mutant efficiently.

Vero cells are known to be good substrate for the production of viruses for pharmaceutical purposes, thus it would be advantageous to have a Vero cell line expressing ICP0 that stably expresses the ICP0 protein for multiple passages and that is capable of complementing ICP0 deficient HSV. Such cells would be suitable for large scale clinical manufacturing of HSV virus vaccines. It is also desirable to have a highly stable Vero cell line that can both efficiently complement ICP0 deficient HSV virus, and inhibit expression of mutant forms of HSV genes that are capable of blocking HSV replication, e.g. dominant negative UL9, and immediate-early expressing of HSV late gene products, e.g. gD.

### SUMMARY OF THE INVENTION

We have now identified a means to generate Vero cell lines that stably express HSV ICP0 protein and that are efficient at complementing ICP0 deficient HSV viruses over multiple cell passages. These cells lack toxic effects of ICP0 as observed in other methods for generation of such cells. We have also established a Vero cell-derived cell line, named V0R-124, that is highly stable at expressing both tetracycline Repressor (tetR) and ICP0 protein. This cell line is capable of maintaining complementation efficiency of ICP0 deficient HSV after extensive cell passage. We were able to generate V0R-124, as well as other Vero cell lines, by the establishment of a promoter-ICP0 expression system in Vero cells that allows for just enough expression of ICP0 to efficiently complement replication of ICP0 deficient HSV, but not for so much expression that it has detrimental effect on cells, e.g. slow cell growth, morphology changes, cell death, and eventual loss of expression due to ICP0 toxic effects. With use of the minimal promoter we were able to generate a parental Vero cell line (e.g. V0-584) that stably expresses ICP0 and that maintains its' ICP0 complementation efficiency for greater than 20 cell passages (even for over 40 cell passages). Because of the cells stability, we were then further able to modify this cell line to generate a cell line that expresses both ICP0 and tetR (e.g V0R-124). We further demonstrate that CJ2-gD2 as well as its derivative replicate efficiently in V0R-124 cells, maintaining its complementation efficiency for greater than 50 cell passages Thus, V0R-124 cells, and other Vero cell lines made using our promoter system for expression of ICP0 in Vero cells, are useful for pharmacologic large scale production of ICP0 deficient HSV, e.g. CJ2-gD2, or its derivatives.

Accordingly, aspects and embodiments of the invention are based upon the establishment of a promoter system for HSV ICP0 expression in Vero cells which allows for successful generation of Vero cell lines that stably express ICP0 at levels just enough to efficiently complement replication of ICP0 deficient HSV virus, however not so much that there is a detrimental loss of expression or cell death due to the toxic effects of ICP0. Embodiments of the invention are also based on the successful generation of Vero cells expressing ICP0 that further comprises a Tet repressor (tetR) gene. These cells are highly efficient at replicating HSV ICP0 deletion virus which also encodes a mutant form of HSV protein that blocks HSV DNA replication e.g. dominant negative HSV UL9. Each of these cells unexpectedly stably express ICP0 even over 20, over 30, and even over 40 cell passages and also efficiently complement HSV ICP0 deficient virus over extensive cell passaging. Such cells are amenable for use in clinical production of vaccines, e.g. HSV vaccines or HSV ICP0 deletion mutant-based oncolytic viruses for tumor therapy

In one aspect of the invention, a Vero cell line is provided that comprises a nucleotide sequence encoding for Herpes Simplex Virus (HSV) ICP0 protein which is operably linked to the HCMV minimal promoter, where the minimal promoter comprises SEQ ID NO: 1. In one embodiment, the promoter further comprises VP16 responsive elements at the 5′ end of the minimal promoter, e.g. SEQ ID NO: 2. In one embodiment, the promoter consists essentially of SEQ ID NO: 2.

In certain embodiments, the HSV ICP0 is selected from HSV-1 ICP0, or variant thereof, and HSV-2 ICP0, or variant thereof. In one embodiment the nucleotide sequence encodes HSV-1 ICP0 that comprises the amino acid sequence of SEQ ID NO: 5. In one embodiment, the nucleotide sequence encodes HSV-2 ICP0 that comprises the amino acid sequence of SEQ ID NO: 6.

In certain embodiments, the Vero cell line comprising a nucleotide sequence that encodes for Herpes Simplex Virus (HSV) ICP0 (e.g. a HSV-1 ICP0 protein or a HSV-2 ICP0 protein) operably linked to a promoter, e.g. a promoter comprising SEQ ID NO: 1 or SEQ ID NO: 2, exhibits the same Vero cell morphology as the cell line from which it was derived (i.e. the parental Vero Cell line morphology). In certain embodiments, the cell line is capable of maintaining ICP0 complementation efficiency, e.g. complementation efficiency is substantially the same whether or not the cell line has been passaged 20, 30, 40, or 50 times within 1 or within 2 standard deviations as measured by a viral replication assay, e.g. plaque forming efficiency assay, an ELISA, or another assay known in the art, for greater than 20 cell passages, or for greater than 30 cell passages, or for even for greater than 40 cell passages.

In another aspect of the invention, a Vero cell line is provided that comprises a nucleotide sequence that encodes for Herpes Simplex Virus (HSV) ICP0 protein, wherein the nucleotide sequence comprises SEQ ID NO: 4. In one embodiment, the Vero cell line exhibits the same Vero cell morphology as the cell line from which it was derived (the parental Vero Cell line morphology). In certain embodiments, the Vero cell line is capable of maintaining ICP0 complementation efficiency, e.g. within 1 or within 2 standard deviations as measured by a viral replication assay, e.g. a plaque forming efficiency assay, ELISA, or other assay, for greater than 20 cell passages, or for greater than 30 cell passages, or even for greater than 40 cell passages.

In another aspect of the invention, a Vero cell line is provided that comprises a nucleotide sequence that encodes for a Herpes Simplex Virus (HSV) ICP0 protein, wherein the nucleotide sequence consists essentially of SEQ ID NO: 4. In one embodiment, the cell line exhibits the same cell morphology as the cell line from which it was derived, i.e. Vero cell morphology. In certain embodiments, the cell line is capable of maintaining ICP0 complementation efficiency, e.g. within 1 or within 2 standard deviations as measured by a viral replication assay, e.g. a plaque forming efficiency assay, an ELISA, or another assay known in the art, for greater than 20 cell passages, or for greater than 30 cell passages, or even for greater than 40 cell passages.

In certain embodiments of each of these aspects of the invention, the Vero cell line further comprises an additional nucleic acid that encodes a tetracycline repressor protein (tetR) operably linked to a promoter. In one embodiment, the promoter comprises SEQ ID NO: 9. In certain embodiments, the nucleic acid that encodes a tetracycline repressor protein encodes the amino acid sequence of SEQ ID NO: 11, or variant thereof. In certain embodiments, the nucleic acid that encodes a tetracycline repressor protein (tetR) which is operably linked to a promoter comprises the nucleotide sequence of SEQ ID NO: 7. This sequence includes a beta-globin intron inserted between the promoter and the tetR nucleotide sequence that encodes the tetR protein. In certain embodiments, the nucleic acid that encodes a tetracycline repressor protein (tetR) operably linked to a promoter consists essentially of SEQ ID NO: 7.

In one aspect, the Vero cell line named V0-584 is provided. V0-584, deposited with the American Type Culture Collection on Apr. 20, 2017 on behalf of The Brigham and Woman's Hospital, Inc. of 75 Francis Street Boston, MA and was assigned ATCC accession no. PTA-124106. We note that reference to this deposit should not create any presumption that such material is necessary to satisfy 35 U.S.C. 112 or that deposit in accordance with these regulations is, or was, required.

In one aspect, the Vero cell line named V0R-124 is provided. V0R-124, deposited with the American Type Culture Collection on Apr. 20, 2017 on behalf of The Brigham and Woman's Hospital, Inc. and was assigned ATCC accession no. PTA-124105. Reference to this deposit should not create any presumption that such material is necessary to satisfy 35 U.S.C. 112 or that deposit in accordance with these regulations is, or was, required.

In certain embodiments of each of these aspects, the Vero cell line further comprises a gene encoding antibiotic resistance. Antibiotic resistant genes are well known to those of skill in the art, including e.g. neomycin and the like.

In certain embodiments of each of these aspects, the Vero cell line further comprises an ICP0 deficient virus, e.g. is infected with an ICP0 deficient virus, or comprises components of an ICP0 deficient virus.

In certain embodiments of each of these aspects, the Vero cell line further comprises a nucleic acid encoding a recombinant protein of interest that is operably linked to a promoter.

In another aspect of the invention, a method of using these Vero cell lines to produce a recombinant protein of interest that is operably linked to a promoter is provided. The recombinant protein may be a therapeutic protein, or a protein for use in a vaccine.

In another aspect of the invention, a method of using these Vero cell lines to produce a virus is provided, e.g. an adenovirus or a Herpes Simplex Virus (HSV). In certain embodiments, the virus is a viral vaccine. In some embodiments, the viral vaccine or viral construct is an ICP0 deficient HSV virus.

In another aspect of the invention, a method of using these Vero cell lines to produce recombinant ICP0 deficient Herpes Simplex Virus (HSV) is provided. The method comprises propagating an ICP0 deficient HSV virus in a Vero cell line of any of each of these aspects of the invention. In one embodiment, the method comprises i) infecting the Vero cell line with an ICP0 deficient HSV virus, ii) incubating the cell line in a tissue culture medium; and iii) collecting the ICP0 deficient virus produced by the cell line.

In another aspect of the invention, methods for generating a Vero cell line that stably expresses HSV ICP0 protein and that efficiently complements ICP0 deficient HSV virus for greater than 20 passages (or even greater than, 30 or 40 passages) is provided. The method comprises contacting Vero cells with a nucleic acid that encodes HSV ICP0 (e.g. HSV1 or HSV2 ICP0) operably linked to a promoter, e.g. wherein the promoter comprises SEQ ID NO: 1 or SEQ ID NO: 2, and screening for complementation of HSV ICP0 deficient virus. In one embodiment, the promoter consists essentially of SEQ ID NO: 2. In one embodiment, the method comprises contacting Vero cells with a nucleic acid that comprises SEQ ID NO: 4. In one embodiment, the HSV ICP0 is HSV-1 ICP0, e.g. SEQ ID NO 5, or variant thereof. In one embodiment, the HSV ICP0 is HSV-2 ICP0, e.g. SEQ ID NO 6, or variant thereof. In one embodiment, the method comprises contacting Vero cells with a nucleic acid that consists essentially of SEQ ID NO: 4 and screening for complementation (replication efficiency) of HSV ICP0 deficient virus.

### BRIEF DESCRIPTION OF FIGURES

FIG. 1 is a graph that shows plaque-forming efficiency of N2-lacZ, an HSV-2 ICP0 null mutant, in Vero cells, V0-584 cells and U2OS cells. Vero cells and V0-584 cells were seeded at 6×10e5 cells per 60 mm dish and U2OS cells were seeded at 1.25×10⁶ cells per 60 mm dish. At 46-48 h post-seeding, triplicate dishes of U2OS cells, V0-584 cells, and Vero cells were infected with N2-lacZ at various PFU/dish. After 1.5 h incubation at 37° C. in an inoculation volume of 0.5 ml/dish, methylcellulose was added. Plaques were stained with neutral-red at 72 h post-infection and counted a day later. The number of input PFU was based on the titer on U2OS cell monolayers.

FIG. 2 is a graph that shows the plaque-forming efficiency of CJ2-gD2 in V0-584 cells, V0R-124 cells and U2CEP4R-11 cells. V0-584 cells and V0R-124 were seeded at 6×10⁵ cells per 60 mm dish and U2CEP4R-11 cells were seeded at 1.25×10⁶ cells per 60 mm dish. At 46-48 h post-seeding, triplicate dishes of U2CEP4R-11 cells, V0-584 cells and V0R-124 cells were infected with CJ2-gD2 at various PFU/dish. After 1.5 h incubation at 37° C. in an inoculation volume of 0.5 ml/dish, methylcellulose was added. Plaques were stained with neutral-red at 72 h post-infection and counted a day later. The number of input PFU was based on the titer on U2CEP4R-11 cell monolayers.

FIG. 3 is a light microscope photograph panel that indicates V0R-124 cells are morphologically similar to Vero cells. Vero cells and V0R-124 cells were seeded at 6×10⁵ cells per 60 mm dish. Cells were photographed under the light microscope at 48 h post-seeding.

FIG. 4 is a graph of plaque-forming efficiency of CJ2-gD2 in V0R-124 cells in either the absence or presence of tetracycline. V0R-124 were seeded at 6×10⁵ cells per 60 mm dish. At 45 h post-seeding, triplicate dishes of V0R-124 cells were infected with CJ2-gD2 at 150 PFU/dish for plaque assay in the absence of doxycycline and at 1.5×10⁴ PFU/dish for plaque-assay in the presence of doxycycline. Plaques were stained with neutral-red at 72 h post-infection and counted a day later. The number of input PFU was based on the titer on V0R-124 cell monolayers.

FIG. 5 is a fluorescent microscope photo panel that indicate regulation of eGFP expression from the tetO-containing HCMV major immediate-early promoter in V0R-124 cells in the absence or the presence of doxycycline. V0R-124 cells and Vero cells were seeded at 5×10⁵ cells per 60 mm dish. At 21 h post-seeding, duplicate dishes of V0R-124 cells and Vero cells were transfected with 0.1 ug/dish of pCDNA4TO-eGFP and 1.5 ug/dish of pCDNA3 by Lipofectinamine 2000. Transfection medium was removed at 4 h post-transfection followed by addition of growth medium with or without doxycycline. Expression of EGFP was photographed under the fluorescence microscopy at 24 h and 48 h post-transfection.

### DESCRIPTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The practice of embodiments of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Current Protocols in Immunology (J. E. Coligan et al., eds., 1999, including supplements through 2016); Current Protocols in Molecular Biology (F. M. Ausubel et al., eds., 1987, including supplements through 2016); Short Protocols in Molecular Biology, F. M. Ausubel et al., eds., fifth edition 2002, including supplements through 2016; Molecular Cloning: A Laboratory Manual, third edition (Sambrook and Russel, 2001); PCR: The Polymerase Chain Reaction, (Mullis et al., eds., 1994); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993), Harlow and Lane Using Antibodies: A Laboratory Manual Cold Spring Harbor Laboratory Press, Cold Spring Harbor, N.Y., 1999; and Beaucage et al. eds., Current Protocols in Nucleic Acid Chemistry John Wiley & Sons, Inc., New York, 2000, including supplements through 2016).

As used herein, the term “cell line” refers to a tissue cultured cell that has been cloned and can be represented by a single cell or a cell population propagated from the single cell clone.

As used herein the term “Vero cell line” refers to a cell line derived from the kidney of a normal adult African green monkey. For example, on Mar. 27, 1962, a cell line derived from the kidney of a normal adult African green monkey named CCL-81 was isolated by Y. Yasumura and Y. Kawakita at the Chiba University in Chiba, Japan; ATCC® CCL-81™ (Manassas, VA 20110 USA). An optional growth medium for a Vero cell line is Sigma's Delbecco's modified Eagle's Medium (Sigma, ST. LOUIS, MO, 63103, USA), Catalog No. D5796. Complete growth medium, is made by adding the following components to the base medium: fetal bovine serum to a final concentration of 10%. Cells are passaged normally, which is by cell dilution at a time when there is about 100% confluence in a tissue culture dish.

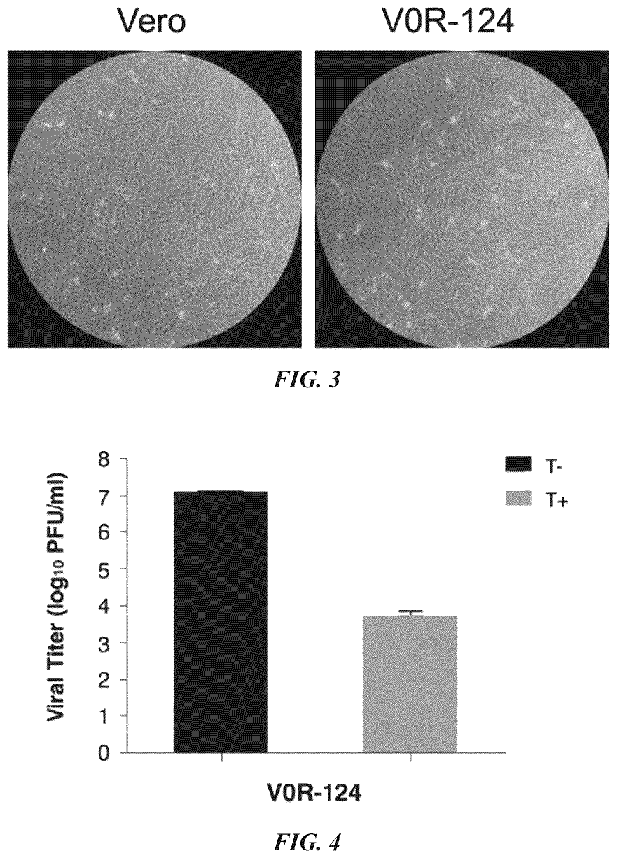

As used herein “Vero cell line morphology” refers to the phenotypic shape and membrane structure of a Vero cell which can be determined by microscope (see for example FIG. 3, that shows the morphology of Vero Cells by light microscope).

As used herein the term “herpes simplex virus” (HSV) refers to both HSV type 1 and HSV type 2. See e.g. Fatahzadeh Ml, Schwartz R A. Human herpes simplex virus infections: epidemiology, pathogenesis, symptomatology, diagnosis, and management, J Am Acad Dermatol. 2007 November; 57 (5): 737-63, ATCC holdings (Manassas, VA 20110 USA) include a number of HSV-1 and HSV-2 strains, including for example: HSV-1 HF; HSV-1 Macintyre; HSV-1 KOS; HSV-1 GHSV-UL46; HSV-1 ATCC-2011-9; HSV-2 MS; HSV-2 G; HSV-2 ATCC-2011-2.

As used herein, the term “ICP0 protein” refers to the HSV protein that is an immediate-early protein which possesses E3 ubiquitin ligase activity. ICP0 activates HSV-1 gene expression, disrupts nuclear domain (ND) 10 structures, mediates the degradation of cellular proteins, and enables evasion of the host's antiviral defenses. As used herein the term “ICP0 deficient HSV” refers to a recombinant HSV vector whose genome does not encode active ICP0 or fully functional ICP0, i.e. ICP0 with normal wild type function. Activity of ICP0 can be monitored using any of the means known to those in the art, See e.g. Miles C Smith et al, HSV-1 ICP0: paving the way for viral replication Future Virol. 2011 April; 6 (4): 421-429; Mirna P Lanfranca et al., HSV-1 ICP0: An E3 Ubiquitin Ligase that counteracts host intrinsic and immunity, Cells 2014 3:438-454.

There are many variants of HSV ICP0 protein, e.g. some of HSV-1 ICP0, strain KOS variants are: Genebank Accession: P08393.1 GI: 124134; Accession: AFI23590.1 GI: 384597746; Accession: AFI23649.1 GI: 384597805; Accession: AFE62827.1 GI: 380776964; Accession: AFE62886.1 GI: 380777023; Accession: ADM22381.1 GI: 304318198; Accession: ALO18731.1 GI: 952947655; Accession: ALO18672.1 GI: 952947596; Accession: ALO18655.1 GI: 952947578; Accession: ALO18596.1 GI: 952947519; Accession: AKH80472.1 GI: 822581062; Accession: AKH80399.1 GI: 822580988; Accession: AKG61929.1 GI: 820021112; Accession: AKG61857.1 GI: 820021035; etc. and the like. Each strain of HSV1 or of HSV2 have multiple variants, all with functional ICP0. These variants are well known in the art and can be found in protein databases. Such variants may be used in methods of the invention. Examples of HSV-2 ICP0 variants, include but are not limited to: Accession: YP_009137210: YP_009137210.1 GI: 820945210; Accession: YP_009137151.1 GI: 820945151; Accession: AEV91397.2 GI: 556197555; Accession: AEV91338.2 GI: 556197550; Accession: ADG01890.1 GI: 295322885; Accession: ADG01889.1 GI: 295322883; Accession: ADG01888.1 GI: 295322881; Accession: ADG01887.1 GI: 295322879; Accession: ADG01885.1 GI: 295322875; Accession: ADG01886.1 GI: 295322877; etc, and the like.

As used herein, the term “variant” or in the context of polypeptides or proteins refers to a polypeptide or protein that comprises an amino acid sequence which has been altered by the introduction of amino acid residue substitutions, deletions and/or additions. Typically substitutions are conservative amino acid substitutions, however non-conservative substitutions can be made that do not destroy the functionality of the protein, e.g. HSV ICP0. “Conservative amino acid substitutions” refers to replacing one amino acid with another having similar structural and/or chemical properties, e.g. such as the replacement of a leucine with an isoleucine or valine, an aspartate with a glutamate, or a threonine with a serine, or glycine with another small amino acid residue. Conservative substitution tables providing functionally similar amino acids are well known in the art. As used herein, the term “non-conservative” refers to substituting an amino acid residue for a different amino acid residue that has different chemical properties. The non-conservative substitutions include, but are not limited to aspartic acid (D) being replaced with glycine (G); asparagine (N) being replaced with lysine (K); or alanine (A) being replaced with arginine (R). For purposes of embodiments of the invention non-conservative substitutions may reduce but does not destroy the proteins normal function.

As used herein the term “comprising” or “comprises” is used in reference to compositions, methods, and respective component(s) thereof, that are essential to the invention, yet open to the inclusion of unspecified elements, whether essential or not.

As used herein the terms, “consisting essentially of,” or variations such as “consists essentially of”, or “consist essentially of” refer to the inclusion of any recited elements, or group of elements, and the optional inclusion of other elements, of similar or different nature than the recited elements, that do not materially change the basic properties of the claimed elements. For example, a nucleotide sequence that consists essentially of a recited sequence may also include additional one or more nucleic acid additions, deletions, or substitutions that do not materially change by a statistically significant amount the expression level of ICP0 and the ability of the ICP0 protein to complement HSV replication at high efficiency. For example, substitutions may correlate to the degenerative amino acid code.

The term “consisting of” refers to compositions, methods, and respective components thereof as described herein, which are exclusive of any element not recited in that description of the embodiment. For example, the nucleotide sequence has no additions, deletions or substitutions.

As used herein, the terms “protein” are used interchangeably and refer to a polymer or oligomer of consecutive amino acid residues.

As used herein, the terms “nucleotide sequence” refers to DNA molecule sequences (e.g., cDNA or genomic DNA.

As used herein, the term “promoter” refers to regulatory control nucleic acid sequences involved in transcription of nucleotide coding sequences, which may or may not include enhancer elements. Such a promoter may be inducible or constitutive. The term “operably linked” refers to a juxtaposition wherein the components described are in a relationship permitting them to function in their intended manner. A promoter “operably linked” to a coding sequence is ligated in such a way that expression of the coding sequence is achieved.

Other than in the operating examples, or where otherwise indicated, all numbers expressing quantities of ingredients or reaction conditions used herein should be understood as modified in all instances by the term “about.” The term “about” when used in connection with percentages may mean ±10%, or even e.g. ±20%, preferably ±10%, more preferably ±5%, still more preferably ±1%. In addition, the singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. The abbreviation, “e.g.” is derived from the Latin exempli gratia, and is used herein to indicate a non-limiting example. Thus, the abbreviation “e.g.” is synonymous with the term “for example.”

The term “statistically significant” or “significantly” refers to statistical significance and generally means a two standard deviation (2SD) above or below a normal or reference level. The term refers to statistical evidence that there is a difference. The decision is often made using the p-value. If within two standard deviations than there is not a statistically significant difference.

Embodiments of the invention are based, in part, upon the surprising successful generation of a Vero cell line that encodes ICP0 protein at levels able to complement ICP0 deficient HSV replication at high efficiency, yet at levels that the ICP0 protein remains stably expressed for greater than 20, greater than 30, and even greater than 40 passages. The stability and low toxicity of expression of the ICP0 protein while still being able to complement ICP0 deficient HSV replication at high efficiency (e.g. as evidenced by plaque formation assay) is quite surprising. In particular, we have identified a promoter system that enables the optimal balance of ICP0 expression in Vero cells. The promoter system uses a promoter that comprises SEQ ID NO: 1.

In certain embodiments, the promoter comprises the addition of VP16 responsive elements, e.g. one, two or three VP16 responsive elements, e.g. see SEQ ID NO: 2.

In particular, cell lines can now be produced that complement replication of ICP0 deficient HSV at high efficiency for greater than 20, greater than 30 and even greater than 40 passages.

In embodiments of the invention efficiency of complementation can be determined using any HSV viral replication assay known to those of skill in the art. One such assay is a plaque forming assay also referred to as a plaque forming efficiency assay. HSV plaque assays determine the number of plaque forming units (pfu) in a virus sample, which is one measure of virus quantity and replication, e.g. if measuring the amount of virus produced by a particular Vero cell line. For example, a confluent monolayer of host cells can be infected with HSV ICP0 deficient virus, e.g. produced from a Vero Cell line described herein, at varying dilutions and covered with a semi-solid medium, such as methylcellulose or agar, to prevent the virus infection from spreading indiscriminately. A viral plaque is formed when a virus infects a cell within the fixed cell monolayer. The virus infected cell will lyse and spread the infection to adjacent cells where the infection-to-lysis cycle is repeated. The infected cell area will create a plaque (an area of infection surrounded by uninfected cells) which can be seen by neutral-red staining or with an optical microscope. Plaque formation can take 3-5 days, depending on the virus being analyzed. Plaques are generally counted manually and the results, in combination with the dilution factor used to prepare the plate, are used to calculate the number of plaque forming units per sample unit volume (pfu/mL). The pfu/mL result represents the number of infective particles within the sample and is based on the assumption that each plaque formed is representative of one infective virus particle. Other assays to monitor replication efficiency include but are not limited to, e.g. a focus forming assay (FFA), protein assays, enzyme-linked immunosorbent assay (ELISA), quantitative polymerase chain reaction (qPCR), and flow cytometry, such assays are well known in the art (e.g. Kaufmann, S. H.; Kabelitz, D. (2002). Methods in Microbiology Vol. 32: Immunology of Infection; Martin, S. (1978). The Biochemistry of Viruses. Cambridge University Press; Flint, S.; Enquist, W.; Racaniello, V. R.; Skalka, A. M. (2009). Virological Methods. Principles of Virology. ASM Press). The HSV replication assay (e.g. an assay to determine how much virus is produced by the Vero cell) that is used to determine ICP0 complementation efficiency of an ICP0 expressing Vero cell line described herein, can include a control reference for High efficiency of ICP0 complementation, such as a control cell line, e.g. U2OS or U2CEP4R-11 cells are known to produce infectious virus at concentration (titer) of 7-8 log₁₀ PFU/ml. For comparison the control U2OS cell line value should be seeded to normalize cell number taking inconsideration the growth rates of the cells as to represent the same number of cells producing virus as the tested Vero cell line. As used herein, “high efficiency” refers to having a plaque-forming efficiency for ICP0 null mutants that is equal to, or less than, that observed with U2OS cells, but no more than 10 fold less than that observed with U2OS cells. In one embodiment, the plaque forming efficiency for ICP0 null mutants in the Vero cell line is no more than 5 fold less, or 4 fold less, or 2 fold less, than what is observed with U2OS cells. U2OS cells are available from the American Type Culture Collection (ATCC); (U-2 0S ATCC® HTB96™).

In certain embodiments, the Vero cell line that stably expresses ICP0 has a parental Vero cell morphology. Cell lines may be visualized by light microscopy to assess morphology, see for example FIG. 3. Cell growth kinetics can also be monitored. In one embodiment, the cell growth kinetics of the Vero cell line expressing ICP0 has the same or close to growth kinetics as the parental Vero cells.

In certain embodiments, the HSV ICP0 is selected from HSV-1 ICP0, or variant thereof, and HSV-2 ICP0, or variant thereof. In one embodiment, the nucleotide sequence encodes HSV-1 ICP0, or variant thereof. In one embodiment the nucleotide sequence encodes HSV-1 ICP0 that comprises the amino acid sequence of SEQ ID NO: 5. In one embodiment, the nucleotide sequence encodes HSV-2 ICP0 that comprises the amino acid sequence of SEQ ID NO: 6.

In one aspect, a Vero cell line is provided that comprises a nucleotide sequence that encodes for Herpes Simplex Virus (HSV) ICP0 protein, wherein the nucleotide sequence comprises SEQ ID NO: 4. In one embodiment, the Vero cell line exhibits the same Vero cell morphology as the cell line from which it was derived (the parental Vero Cell line morphology). In certain embodiments, the Vero cell line is capable of maintaining ICP0 complementation efficiency, e.g. within 1 or within 2 standard deviations as measured by plaque forming efficiency assay, for greater than 20 cell passages, or for greater than 30 cell passages, or for even for greater than 40 cell passages.

In one aspect, a Vero cell line is provided that comprises a nucleotide sequence that encodes for a Herpes Simplex Virus (HSV) ICP0 protein e.g. a HSV-1 ICP0 protein or a HSV-2 ICP0 protein, operably linked to a promoter wherein the promoter comprises SEQ ID NO: 2.

In one aspect, a Vero cell line is provided that comprises a nucleotide sequence that encodes for a Herpes Simplex Virus (HSV) ICP0 protein e.g. a HSV-1 ICP0 protein or a HSV-2 ICP0 protein, operably linked to a promoter wherein the promoter consists essentially of SEQ ID NO: 2.

In one aspect, a Vero cell line is provided that comprises the nucleotide sequence of SEQ ID NO: 4, which is a sequence that encodes for a HSV-1 ICP0 protein operably linked to the promoter of SEQ ID NO: 2.

In certain embodiments of each of these aspects, the Vero cell line further comprises an additional nucleic acid that encodes a tetracycline repressor protein (tetR) operably linked to a promoter.

As used herein, a tetracycline repressor protein (tetR) refers to a transcriptional repressor protein that regulates transcription (See e.g Ramos, Juan L et al. (2005-06-01). “The TetR Family of Transcriptional Repressors”. Microbiology and Molecular Biology Reviews 69 (2): 326-356, 2005). Many tetR proteins are known in the art and are suitable for use in embodiments of the invention. In one embodiment, the nucleic acid that encodes a tetracycline repressor protein that encodes the amino acid sequence of SEQ ID NO: 11, or variant thereof.

Any promoter known to those of skill in the art may be operably linked to the TetR nucleic acid in embodiments of the invention. In one embodiment, the promoter operably linked to the nucleic acid encoding tetR, comprises SEQ ID NO: 9. In certain embodiments, a beta-globin intron is inserted between the promoter and the tetR nucleotide sequence that encodes the tetR protein (of SEQ ID NO: 7).

In another aspect of the invention, a method of using these Vero cell lines to produce recombinant ICP0 deficient Herpes Simplex Virus (HSV) is provided. The method comprises propagating an ICP0 deficient HSV virus in a Vero cell line of any of each of these aspects of the invention. In one embodiment, the method comprises i) infecting the Vero cell line with an ICP0 deficient HSV virus, ii) incubating the cell line in a tissue culture medium; and iii) collecting the ICP0 deficient virus produced by the cell line. Methods for producing and isolation HSV virus are well known to those of skill in the art, See e.g. Goins WF1, et al. Construction and production of recombinant herpes simplex virus vectors, Methods Mol Biol. 2008; 433:97-113; and Herpes Simplex Virus: Propagation, Quantification, and Storage, Current protocols in microbiology by John Wiley & Sons, Inc. Chapter 14, 14. E.1-14E.23, contributed by John A. Blaho, Elise R. Morton, and Jamie C. Yedowitz, October 2005). The Vero cell lines described herein use the same medium as suggested for Vero cells.

Methods for generating Vero cell lines that stably expresses HSV ICP0 protein and that efficiently complements ICP0 deficient HSV are also provided. The methods comprise contacting Vero cells with a nucleic acid that encodes HSV ICP0 (e.g. HSV1 or HSV2 ICP0) operably linked to a promoter, e.g, wherein the promoter comprises SEQ ID NO: 1 or SEQ ID NO: 2, and screening for complementation of HSV ICP0 deficient virus. Standard transfection protocols known in the art can be used for contacting the Vero cells with the nucleic acids described herein. See e.g. Current Protocols in Molecular Biology (F. M. Ausubel et al., eds., 1987, including supplements through 2016). Complementation efficiently can be monitored using any HSV replication efficiency assay known to those in the art, e.g. a plaque forming assay or ELISA assay.

In one embodiment, the method comprises contacting Vero cells with a nucleotide sequence that encodes for a Herpes Simplex virus (HSV) ICP0 protein operably linked to a promoter, wherein the promoter comprises SEQ ID NO:1. In one embodiment, the method comprises contacting Vero cells with a nucleotide sequence that encodes for a Herpes Simplex virus (HSV) ICP0 protein operably linked to a promoter, wherein the promoter comprises SEQ ID NO:2. In one embodiment, the method comprises contacting Vero cells with a nucleotide sequence that encodes for a Herpes Simplex virus (HSV) ICP0 protein that is operably linked to a promoter, wherein the promoter consists essentially of SEQ ID NO: 2. In one embodiment, the HSV ICP0 is HSV-1 ICP0, e.g. SEQ ID NO 5, or variant thereof. In one embodiment, the HSV ICP0 is HSV-2 ICP0, e.g. SEQ ID NO 6, or variant thereof.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Embodiments of the invention are further described in the following numbered paragraphs.
1. A Vero cell line comprising a nucleotide sequence that encodes for a Herpes Simplex virus (HSV) ICP0 protein operably linked to a promoter, wherein the promoter comprises SEQ ID NO:1.
2. The Vero cell line of paragraph 1, wherein the promoter comprises SEQ ID NO: 2.
3. The Vero cell line of any one of paragraphs 1-2, wherein the nucleotide sequence encodes for HSV-1 ICP0 that comprises the amino acid sequence of SEQ ID NO: 5.
4. The Vero cell line of any one of paragraphs 1-3, wherein, the cell line exhibits Vero cell morphology.
5. The Vero cell line of any one of paragraphs 1-4, wherein the cell line is capable of maintaining ICP0 complementation efficiency within 2 standard deviations as measured by a viral replication assay for greater than 20 cell passages.
6. The Vero cell line of any one of paragraphs 1-5, wherein the cell line is capable of maintaining ICP0 complementation efficiency within 2 standard deviations as measured by a viral replication assay for greater than 30 cell passages.
7. The Vero cell line of any one of paragraphs 1-6, wherein the cell line is capable of maintaining ICP0 complementation efficiency within 2 standard deviations as measured by a viral replication assay for greater than 40 cell passages.
8. The Vero cell line of any one of paragraphs 1-7, wherein the nucleotide sequence that encodes for a Herpes Simplex virus (HSV) ICP0 protein operably linked to a promoter comprises SEQ ID NO: 4.
9. The Vero cell line of any one of paragraphs 1-7, wherein the nucleotide sequence that encodes for a Herpes Simplex virus (HSV) ICP0 protein operably linked to a promoter consists essentially of SEQ ID NO: 4.
10. The Vero cell line of any one of paragraphs 1-9, wherein the cell line further comprises an additional nucleic acid that encodes a tetracycline repressor protein (tetR) operably linked to a promoter.
11. The Vero cell line of paragraph 10, wherein the promoter operably linked to the tetracycline repressor protein comprises SEQ ID NO: 9.
12. The Vero cell line of any one of paragraphs 10-11, wherein the nucleic acid that encodes a tetracycline repressor protein encodes the amino acid sequence of SEQ ID NO: 11.
13. The Vero cell line of any one of paragraphs 10-12, wherein the nucleic acid that encodes tetracycline repressor protein (tetR) operably linked to a promoter comprises SEQ ID NO: 7.
14. The Vero cell line of any one of paragraphs 10-12, wherein the nucleic acid that encodes a tetracycline repressor protein (tetR) operably linked to a promoter consists essentially of SEQ ID NO: 7.
15. The Vero cell line of paragraph 9, which is V0-584.
16. The Vero cell line of paragraph 14, which is V0R-124.
17. The Vero cell line of any one of paragraphs 1-16, further comprising a gene encoding antibiotic resistance.
18. The Vero cell line of any one of paragraphs 1-17, further comprising an ICP0 deficient HSV virus.
19. The Vero cell line of any one of paragraphs 1-17, further comprising a nucleic acid encoding a recombinant protein of interest that is operably linked to a promoter.
20. The Vero cell line of paragraph 19, wherein the recombinant protein of interest is a therapeutic protein.
21. The Vero cell line of paragraph 20, wherein the therapeutic protein is a vaccine protein.
22. A method of producing a viral vaccine of interest comprising propagating a virus to be used for vaccination in a Vero cell line of any one of paragraphs 1-21.
23. The method of paragraph 22, wherein the virus to be used for vaccination is an ICP0 deficient HSV virus.
24. The method of paragraph 22, wherein the virus is an adenovirus.
25. A method of producing a recombinant protein of interest comprising propagating the Vero cell line of any one of paragraphs 19-21, for sufficient time to allow for expression of the protein.
26. A method of producing ICP0 deficient HSV virus comprising propagating an HSV ICP0 deficient HSV virus in a Vero cell line of paragraph 1.
27. A method of producing ICP0 deficient HSV virus comprising i) infecting a Vero cell line of any of paragraph 1, with an ICP0 deficient HSV virus, ii) incubating the cell line in a tissue culture medium; and iii) collecting the ICP0 deficient virus produced by the cell line.

All references, publications and patents described herein, in the Examples and throughout the Specification, are incorporated herein by reference in their entirety. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Materials and Methods

Cells: African Green Monkey Kidney CCL-81 (Vero) cells (ATCC) and the human osteosarcoma line U2OS cells were grown and maintained in Dulbecco's Modified Eagle's Medium (DMEM; Sigma Aldrich) supplemented with 10% fetal bovine serum (FBS) in the presence of 100 U/ml penicillin G and 100 μg/ml streptomycin sulfate (GIBCO, Carlsbad, CA) (Yao, et al., J. Virol. 69:6249-58 (1995)). U2OS cells are able to complement functionally for the HSV-1 ICP0 deletion (Yao, et al., J. Virol. 69:6249-58 (1995)). U2CEP4R11 cells are tetR-expressing U2OS cells that were maintained in DMEM plus 10% FBS and hygromycin B at 50 μg/ml (Yao, et al., Hum. Gene Ther. 9:1939-50 (1998)).

Plasmids: pMF3-ICP0 is an HSV-1 ICP0-expressing plasmid that contain no HSV-1 sequence flanking the HSV-1 ICP0 ORF (SEQ ID NO: 3). pMF3-ICP3 encodes ICP0 ORF under the control of modified HCMV minimal promoter consisting of 3 VP16 responsive elements plus 2×CAAAT/SP1 elements 5′ to the HCMV TATA element (SEQ ID NO: 4). pcDNA3 and pCDNA4/TO were obtained from Invitrogen (Carlsbad, CA). pcDNA3 encodes neomycin-resistant gene under the control of SV40 promoter. pCDNA4/TO encodes Zeocin-resistant gene. pCDNA4TO-eGFP is an eGFP (enhanced green fluorescent protein)-expressing plasmid.

Plasmid pMF-tetR contains a synthesized DNA fragment consisting of an optimized tetR transcription unit that includes: 1) a modified HCMV major immediate-early promoter that is lack of HCMV promoter sequence from −174 to −370 bp and contains two HSV-1 VP16 responsive elements with the first VP16 responsive element at 149 bp upstream of the HCMV TATA element and the second VP16 responsive element at 274 bp upstream of the HCMV TATA element, 2) beta-globin intron, and 3) a codon optimized tetR coding sequence followed by SV40 poly A signal sequence (SEQ ID NO: 7).

Viruses: N2-lacZ is an HSV-2 ICP0 null mutant, in which the Xho I-ICP0 coding sequence in both copies of the ICP0 gene in the HSV-2 genome are replaced by the Lac Z gene. CJ2-gD2 is an HSV-2 ICP0-deletion mutant-based non-replicating dominant-negative HSV-2 recombinant virus in which both copies of the lacZ gene in N2-lacZ are replaced by DNA sequences encoding the gD2 gene driven by the tetO-bearing HSV-1 major immediate-early ICP4 promoter, while the gene encoding UL9-C535C is under the control of the tetO-containing hCMV major immediate-early promoter in an opposite orientation of the inserted gD2 gene (Akhrameyeva, J. Virol. 85:5036-47 (2011)). N2-lacZ was propagated in U2OS cells, while CJ2-gD2 was propagated and plaque assayed in U2CEP4R11 cells.

### Example 1: Establishment of an HSV-1 ICP0 Expressing Stable Cell Line that can Complement the Plaque Forming Efficiency of HSV-2 ICP0 Null Mutant Comparable to that of a Human Osteosacarma Cell Line, U2OS Cells

We were able to successfully minimize the cytotoxic effect of ICP0 by using a minimal promoter pMF-3 (SEQ ID NO: 01). To establish the ICP0-expressing stable cell lines, we constructed an ICP0-expressing plasmid, pMF3-ICP0. Linearized pMF3-ICP0 plasmid was then transfected into CCL-81 Vero cells along with linearized pcDNA3 by Lipofectamine 2000 (Invitrogen Inc.). At 30 hours post-transfection, cells were seeded into 100 mm dishes at various cell density and were grown in DMEM growth medium containing G418 at 400 μg/ml. G418 resistant colony cells were assayed by its ability to complement the growth of HSV-2 ICP0 null mutant, N2-lacZ, benchmarked against U2OS cells. Among more than 1000 G418-resistant colonies we selected, V0-584 cells represent the only G418-resistant and ICP0-expressing stable cell line that can complement the plaque-formation of N2-lacZ at level comparable to U2OS cells. The results in FIG. 1 show that the plaque-forming efficacy of N2-lacZ in passage 7 V0-584 cells is ˜2-fold lower than that of U2OS cells and ˜120-fold higher than that of normal CCL-81 cells. The complementing efficiency of V0-584 cells for N2-lacZ remains 2-3 fold lower than that of U2OS cells at passage 33.

### Example 2: Establishment of a tetR- and ICP0-Expressing Vero Cells that can Complement the Plaque Forming Efficiency of CJ2-gD2 Efficiently

We have synthesized a DNA sequence at GeneArt (Invitrogen Inc.), which contains an optimized tetR transcription unit that includes: 1) a modified HCMV major immediate-early promoter, which contain the first VP16 responsive element at position 149 bp and the second VP16 responsive element at position 274 bp upstream of the HCMV TATA element, respectively (SEQ ID NO: 9), 2) beta-globin intron (SEQ ID NO: 10), and 3) a codon optimized tetR coding sequence (SEQ ID NO: 8) followed by SV40 poly A signal sequence. The plasmid that contains above mentioned DNA sequence was named pMF-tetR.

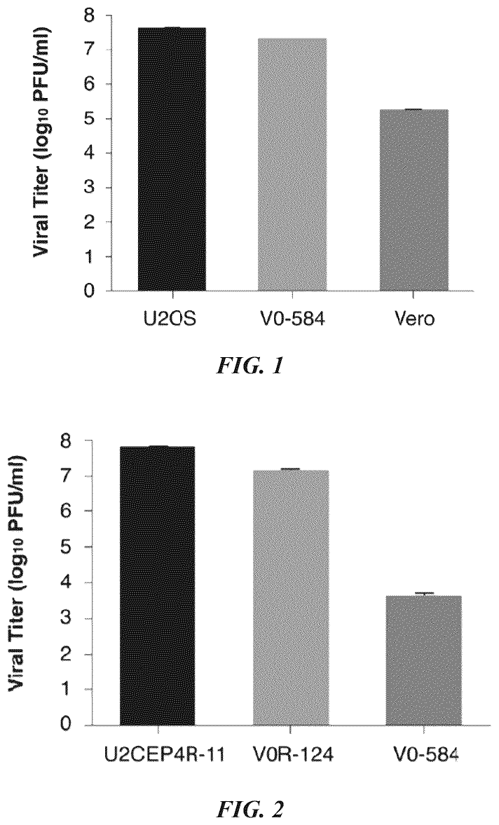

To establish tetR- and ICP0-expressing stable CCL-81 Vero cells, V0-584 cells were transfected with linearized pMF-tetR along with linearized pCDNA4/TO (Invitrogen) by Lipofectamine 2000. Transfected cells were then seeded into 100 mm dishes at various cell density and were grown in DMEM growth medium containing G418 at 400 μg/ml and Zeocin at 200 μg/ml. G418- and Zeocin-resistant colony cells were assayed by its ability to complement the growth of CJ2-gD2, benchmarked against U2CEP4R-11 cells. V0R-124 cells represent a G418/Zeocin-resistant stable cell line that can complement the plaque-formation of CJ2-gD2 efficiently even at passage 41. The results in FIG. 2 show that the plaque-forming efficacy of CJ2-gD2 in V0R-124 cells is ˜ 4.5-fold lower than that of U2CEP4R-11 cells and is 3,378-fold higher than that of V0-584 cells. The plaque-forming efficiency of N2-lacZ on V0R-124 cells is similar to that of V0-584 cells (data not shown). Notably, V0R-124 cells are morphologically similar to CCL-81 cells and exhibit similar growth kinetics as CCL-81 cells (FIG. 3).

FIG. 4 represents the plaque-forming efficiency of CJ2-gD2 in V0R-124 cells in either the absence or presence of doxycycline. The result shows that the plaque-forming efficiency of CJ2-D2 in V0R-124 cells in the presence of tetracycline is 2,418-fold lower than in the absence of tetracycline, demonstrating further that like in tetR-expressing Vero cell line, VCEP4R-28 cells (Akhrameyeva, J. Virol. 85:5036-47 (2011)), tetR expressed in V0R-124 cells can effectively suppresses the expression of UL9-C535C in CJ2-gD2-infected V0R-124 cells in the absence of doxycycline.

### Example 3: Regulation of Gene Expression from the tetO-Containing HCMV Major Immediate-Early Promoter in V0R-124 Cells

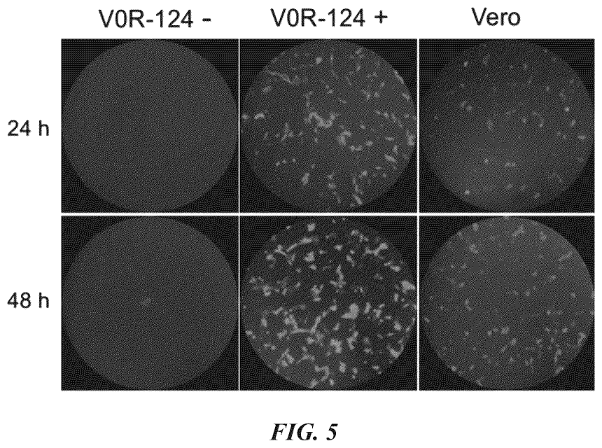

To further examine the effectiveness of tetR expressed in V0R-124 cells in regulating gene expression from the tetO-containing promoters, V0R-124 cells were seeded into 60 mm dishes at 6×10⁵ cells/dish. At 21 h post-seeding, cells were transfected with pCDNA4TO-eGFP with Lipofectamine 2000. Transfection medium was removed at 4 h post-transfection followed by addition of normal growth medium with no tetracycline or with doxycycline at a concentration of 0.2 ug/ml. Expression of EGFP was visualized directly under the fluorescence microscopy at 24 h and 48 h post-transfection. As shown in FIG. 5 while a significant number of green cells were seen in V0R-124 cells grown in the presence of doxycycline, a few faint green cells were detected under un-induced conditions, showing that gene expression of the tetO-containing promoter can be tightly regulated by doxycycline in V0R-124 cells.

Moreover, given the intensity of eGFP detected in transfected Vero cells and V0R-124 cells in the presence of tetracycline, the results suggest further that expression of ICP0 in V0R-124 cells can effectively elevate gene expression following transient transfection, consistent with the previously demonstrated potent trans-activating function of ICP0 on gene expression.

SEQUENCE LISTING

SEQ ID NO: 1 Minimal Promoter Sequence in pMF3-ICP0
CCATTGACGCAAATGGGCGGTAGGCGTGTACGGTGGGAGGTCTATATAAG (SEQ ID NO: 1)
SEQ ID NO: 2 Promoter sequence in pMF3-ICP0 (with 3 x VP16 responsive elements
(underlined and highlight) plus 2 x CAAATGGGCGG cis-acting elements (SEQ ID NO: 12))
used for the expression of HSV-1 ICP0 coding sequence.
ATGCTAATGATATACATGCCACGTACTTATGGTGTCTATGCTAATGATATTCGCAAATGGGC
GGTAGACCGGTGAATTCATGCTAATGATATTCTTTGGTACCATTGACGCAAATGGGCGGTAG
GCGTGTACGGTGGGAGGTCTATATAAG (SEQ ID NO: 2)
SEQ ID NO: 3 HSV-1 ICP0 coding sequence followed by SV40 poly A signal sequence in
plasmids pMF3-ICP0.
ATGGAGCCCCGCCCCGGAGCGAGTACCCGCCGGCCTGAGGGCCGCCCCCAGCGCGAGGTGA
GGGGCCGGGCGCCATGTCTGGGGCGCCATATTGGGGGGCGCCATATTGGGGGGCGCCATGT
TGGGGGACCCCCGACCCTTACACTGGAACCGGCCGCCATGTTGGGGGACCCCCACTCATAC
ACGGGAGCCGGGCGCCATGTTGGGGCGCCATGTTAGGGGGCGTGGAACCCCGTGACACTAT
ATATACAGGGACCGGGGGCGCCATGTTAGGGGGTGCGGAACCCCCTGACCCTATATATACA
GGGACCGGGGTCGCCCTGTTGGGGGTCGCCATGTGACCCCCTGACTTTATATATACAGACCC
CCAACACATACACATGGCCCCTTTGACTCAGACGCAGGGCCCGGGGTCGCCGTGGGACCCC
CTGACTCATACACAGAGACACGCCCCCACAACAAACACACAAGGACCGGGGTCGCCGTGTT
GGGGGCGTGGTCCCCACTGACTCATACGCAGGCCCCCCTTACTCACACGCATCTAGGGGGGT
GGGGAGGAGCCGCCCGCCATATTTGGGGGACGCCGTGGGACCCCCGACTCCGGTGCGTCTG
GAGGGCGGGAGAAGAGGGAAGAAGAGGGGTCGGGATCCAAAGGACGGACCCAGACCACCT
TTGGTTGCAGACCCCTTTCTCCCCCCTCTTCCGAGGCCAGCAGGGGGGCAGGACTTTGTGAG
GCGGGGGGGGGAGAGGGGGAACTCGTGGGTGCTGATTGACGCGGGAAATCCCCCCCCATTC
TTACCCGCCCCCCTTTTTTCCCCTTAGCCCGCCCCGGATGTCTGGGTGTTTCCCTGCGACCGA
GACCTGCCGGACAGCAGCGACTCTGAGGCGGAGACCGAAGTGGGGGGGCGGGGGGACGCC
GACCACCATGACGACGACTCCGCCTCCGAGGCGGACAGCACGGACACGGAACTGTTCGAGA
CGGGGCTGCTGGGGCCGCAGGGCGTGGATGGGGGGGCGGTCTCGGGGGGGAGCCCCCCCC
GCGAGGAAGACCCCGGCAGTTGCGGGGGCGCCCCCCCTCGAGAGGACGGGGGGAGCGACG
AGGGCGACGTGTGCGCCGTGTGCACGGATGAGATCGCGCCCCACCTGCGCTGCGACACCTT
CCCGTGCATGCACCGCTTCTGCATCCCGTGCATGAAAACCTGGATGCAATTGCGCAACACCT
GCCCGCTGTGCAACGCCAAGCTGGTGTACCTGATAGTGGGCGTGACGCCCAGCGGGTCGTT
CAGCACCATCCCGATCGTGAACGACCCCCAGACCCGCATGGAGGCCGAGGAGGCCGTCAGG
GCGGGCACGGCCGTGGACTTTATCTGGACGGGCAATCAGCGGTTCGCCCCGCGGTACCTGA
CCCTGGGGGGGCACACGGTGAGGGCCCTGTCGCCCACCCACCCGGAGCCCACCACGGACGA
GGATGACGACGACCTGGACGACGGTGAGGCGGGGGGCGGCAAGGACCCTGGGGGAGGAGG
AGGAGGAGGGGGGGGGAGGGAGGAATAGGCGGGCGGGCGAGGAAAGGGCGGGCCGGGGA
GGGGGCGTAACCTGATCGCGCCCCCCGTTGTCTCTTGCAGCAGACTACGTACCGCCCGCCCC
CCGCCGGACGCCCCGCGCCCCCCCACGCAGAGGCGCCGCCGCGCCCCCCGTGACGGGCGGG
GCGTCTCACGCAGCCCCCCAGCCGGCCGCGGCTCGGACAGCGCCCCCCTCGGCGCCCATCG
GGCCACACGGCAGCAGTAACACCAACACCACCACCAACAGCAGCGGCGGCGGCGGCTCCC
GCCAGTCGCGAGCCGCGGCGCCGCGGGGGGCGTCTGGCCCCTCCGGGGGGGTTGGGGTTGG
GGTTGGGGTTGTTGAAGCGGAGGCGGGGCGGCCGAGGGGCCGGACGGGCCCCCTTGTCAAC
AGACCCGCCCCCCTTGCAAACAACAGAGACCCCATAGTGATCAGCGACTCCCCCCCGGCCT
CTCCCCACAGGCCCCCCGCGGCGCCCATGCCAGGCTCCGCCCCCCGCCCCGGGCCCCCCGCG
TCCGCGGCCGCGTCGGGACCCGCGCGCCCCCGCGCGGCCGTGGCCCCGTGCGTGCGAGCGC
CGCCTCCGGGGCCCGGCCCCCGCGCCCCGGCCCCCGGGGCGGAGCCGGCCGCCCGCCCCGC
GGACGCGCGCCGTGTGCCCCAGTCGCACTCGTCCCTGGCTCAGGCCGCGAACCAAGAACAG
AGTCTGTGCCGGGCGCGTGCGACGGTGGCGCGCGGCTCGGGGGGGCCGGGCGTGGAGGGTG
GGCACGGGCCCTCCCGCGGCGCCGCCCCCTCCGGCGCCGCCCCGCTCCCCTCCGCCGCCTCT
GTCGAGCAGGAGGCGGCGGTGCGTCCGAGGAAGAGGCGCGGGTCGGGCCAGGAAAACCCC
TCCCCCCAGTCCACGCGTCCCCCCCTCGCGCCGGCAGGGGCCAAGAGGGCGGCGACGCACC
CCCCCTCCGACTCAGGGCCGGGGGGGCGCGGCCAGGGTGGGCCCGGGACCCCCCTGACGTC
CTCGGCGGCCTCCGCCTCTTCCTCCTCTGCCTCTTCCTCCTCGGCCCCGACCCCCGCGGGGGC
CGCCTCTTCCGCCGCCGGGGCCGCGTCCTCCTCCGCTTCCGCCTCCTCGGGCGGGGCCGTCG
GTGCCCTGGGAGGGAGACAAGAGGAAACCTCCCTCGGCCCCCGCGCTGCTTCTGGGCCGCG
GGGGCCGAGGAAGTGTGCCCGGAAGACGCGCCACGCGGAGACTTCCGGGGCCGTCCCCGC
GGGCGGCCTCACGCGCTACCTGCCCATCTCGGGGGTCTCTAGCGTGGTCGCCCTGTCGCCTT
ACGTGAACAAGACTATCACGGGGGACTGCCTGCCCATCCTGGACATGGAGACGGGGAACAT
CGGGGCGTACGTGGTCCTGGTGGACCAGACGGGAAACATGGCGACCCGGCTGCGGGCCGCG
GTCCCCGGCTGGAGCCGCCGCACCCTGCTCCCCGAGACCGCGGGTAACCACGTGATGCCCC
CCGAGTACCCGACGGCCCCCGCGTCGGAGTGGAACAGCCTCTGGATGACCCCCGTGGGGAA
CATGCTGTTCGACCAGGGCACCCTAGTGGGCGCCCTGGACTTCCGCAGCCTGCGGTCTCGGC
ACCCGTGGTCCGGGGAGCAGGGGGCGTCGACCCGGGACGAGGGAAAACAATAACAGAACT
TGTTTATTGCAGCTTATAATGGTTACAAATAAAGCAATAGCATCACAAATTTCACAAATAAA
GCATTTTTTTCACTGCATTCTAGTTGTGGTTTGTCCAAACTCATCAATGTATCTTATCATGTCT
GAAGCTTGGC (SEQ ID NO: 3)
SEQ ID NO: 4 2 Promoter sequence in pMF3-ICP0 (with 3 x VP16 responsive elements
(underlined) plus 2 x CAAATGGGCGG cis-acting elements (SEQ ID NO: 12)) used for the
expression of HSV-1 ICP0 coding sequence plus the HSV-1 ICP0 coding sequence.
ATGCTAATGATATACATGCCACGTACTTATGGTGTCTATGCTAATGATATTCGCAAATGGGC
GGTAGACCGGTGAATTCATGCTAATGATATTCTTTGGTACCATTGACGCAAATGGGCGGTAG
GCGTGTACGGTGGGAGGTCTATATAAGCAGAGCTCGTTTAGTGAACCGTCAGATCGCCTGG
AGACGCCATCCACGCTGTTTTGACCTCCATAGAAGACACCGGGACCGATCCAGCCTCCGCG
GCCGGGAACGGTGCATTGGAACGGACTCTAGAGGATCCATGGAGCCCCGCCCCGGAGCGAG
TACCCGCCGGCCTGAGGGCCGCCCCCAGCGCGAGGTGAGGGGCCGGGCGCCATGTCTGGGG
CGCCATATTGGGGGGCGCCATATTGGGGGGCGCCATGTTGGGGGACCCCCGACCCTTACAC
TGGAACCGGCCGCCATGTTGGGGGACCCCCACTCATACACGGGAGCCGGGCGCCATGTTGG
GGCGCCATGTTAGGGGGCGTGGAACCCCGTGACACTATATATACAGGGACCGGGGGCGCCA
TGTTAGGGGGTGCGGAACCCCCTGACCCTATATATACAGGGACCGGGGTCGCCCTGTTGGG
GGTCGCCATGTGACCCCCTGACTTTATATATACAGACCCCCAACACATACACATGGCCCCTT
TGACTCAGACGCAGGGCCCGGGGTCGCCGTGGGACCCCCTGACTCATACACAGAGACACGC
CCCCACAACAAACACACAAGGACCGGGGTCGCCGTGTTGGGGGCGTGGTCCCCACTGACTC
ATACGCAGGCCCCCCTTACTCACACGCATCTAGGGGGGTGGGGAGGAGCCGCCCGCCATAT
TTGGGGGACGCCGTGGGACCCCCGACTCCGGTGCGTCTGGAGGGCGGGAGAAGAGGGAAG
AAGAGGGGTCGGGATCCAAAGGACGGACCCAGACCACCTTTGGTTGCAGACCCCTTTCTCC
CCCCTCTTCCGAGGCCAGCAGGGGGGCAGGACTTTGTGAGGCGGGGGGGGGAGAGGGGGA
ACTCGTGGGTGCTGATTGACGCGGGAAATCCCCCCCCATTCTTACCCGCCCCCCTTTTTTCCC
CTTAGCCCGCCCCGGATGTCTGGGTGTTTCCCTGCGACCGAGACCTGCCGGACAGCAGCGAC
TCTGAGGCGGAGACCGAAGTGGGGGGGCGGGGGGACGCCGACCACCATGACGACGACTCC
GCCTCCGAGGCGGACAGCACGGACACGGAACTGTTCGAGACGGGGCTGCTGGGGCCGCAG
GGCGTGGATGGGGGGGCGGTCTCGGGGGGGAGCCCCCCCCGCGAGGAAGACCCCGGCAGT
TGCGGGGGCGCCCCCCCTCGAGAGGACGGGGGGAGCGACGAGGGCGACGTGTGCGCCGTG
TGCACGGATGAGATCGCGCCCCACCTGCGCTGCGACACCTTCCCGTGCATGCACCGCTTCTG
CATCCCGTGCATGAAAACCTGGATGCAATTGCGCAACACCTGCCCGCTGTGCAACGCCAAG
CTGGTGTACCTGATAGTGGGCGTGACGCCCAGCGGGTCGTTCAGCACCATCCCGATCGTGAA
CGACCCCCAGACCCGCATGGAGGCCGAGGAGGCCGTCAGGGCGGGCACGGCCGTGGACTTT
ATCTGGACGGGCAATCAGCGGTTCGCCCCGCGGTACCTGACCCTGGGGGGGCACACGGTGA
GGGCCCTGTCGCCCACCCACCCGGAGCCCACCACGGACGAGGATGACGACGACCTGGACGA
CGGTGAGGCGGGGGGCGGCAAGGACCCTGGGGGAGGAGGAGGAGGAGGGGGGGGGAGGG
AGGAATAGGCGGGCGGGCGAGGAAAGGGCGGGCCGGGGAGGGGGCGTAACCTGATCGCGC
CCCCCGTTGTCTCTTGCAGCAGACTACGTACCGCCCGCCCCCCGCCGGACGCCCCGCGCCCC
CCCACGCAGAGGCGCCGCCGCGCCCCCCGTGACGGGCGGGGCGTCTCACGCAGCCCCCCAG
CCGGCCGCGGCTCGGACAGCGCCCCCCTCGGCGCCCATCGGGCCACACGGCAGCAGTAACA
CCAACACCACCACCAACAGCAGCGGCGGCGGCGGCTCCCGCCAGTCGCGAGCCGCGGCGCC
GCGGGGGGCGTCTGGCCCCTCCGGGGGGGTTGGGGTTGGGGTTGGGGTTGTTGAAGCGGAG
GCGGGGCGGCCGAGGGGCCGGACGGGCCCCCTTGTCAACAGACCCGCCCCCCTTGCAAACA
ACAGAGACCCCATAGTGATCAGCGACTCCCCCCCGGCCTCTCCCCACAGGCCCCCCGCGGC
GCCCATGCCAGGCTCCGCCCCCCGCCCCGGGCCCCCCGCGTCCGCGGCCGCGTCGGGACCC
GCGCGCCCCCGCGCGGCCGTGGCCCCGTGCGTGCGAGCGCCGCCTCCGGGGCCCGGCCCCC
GCGCCCCGGCCCCCGGGGCGGAGCCGGCCGCCCGCCCCGCGGACGCGCGCCGTGTGCCCCA
GTCGCACTCGTCCCTGGCTCAGGCCGCGAACCAAGAACAGAGTCTGTGCCGGGCGCGTGCG
ACGGTGGCGCGCGGCTCGGGGGGGCCGGGCGTGGAGGGTGGGCACGGGCCCTCCCGCGGC
GCCGCCCCCTCCGGCGCCGCCCCGCTCCCCTCCGCCGCCTCTGTCGAGCAGGAGGCGGCGGT
GCGTCCGAGGAAGAGGCGCGGGTCGGGCCAGGAAAACCCCTCCCCCCAGTCCACGCGTCCC
CCCCTCGCGCCGGCAGGGGCCAAGAGGGCGGCGACGCACCCCCCCTCCGACTCAGGGCCGG
GGGGGCGCGGCCAGGGTGGGCCCGGGACCCCCCTGACGTCCTCGGCGGCCTCCGCCTCTTC
CTCCTCTGCCTCTTCCTCCTCGGCCCCGACCCCCGCGGGGGCCGCCTCTTCCGCCGCCGGGG
CCGCGTCCTCCTCCGCTTCCGCCTCCTCGGGCGGGGCCGTCGGTGCCCTGGGAGGGAGACAA
GAGGAAACCTCCCTCGGCCCCCGCGCTGCTTCTGGGCCGCGGGGGCCGAGGAAGTGTGCCC
GGAAGACGCGCCACGCGGAGACTTCCGGGGCCGTCCCCGCGGGCGGCCTCACGCGCTACCT
GCCCATCTCGGGGGTCTCTAGCGTGGTCGCCCTGTCGCCTTACGTGAACAAGACTATCACGG
GGGACTGCCTGCCCATCCTGGACATGGAGACGGGGAACATCGGGGCGTACGTGGTCCTGGT
GGACCAGACGGGAAACATGGCGACCCGGCTGCGGGCCGCGGTCCCCGGCTGGAGCCGCCG
CACCCTGCTCCCCGAGACCGCGGGTAACCACGTGATGCCCCCCGAGTACCCGACGGCCCCC
GCGTCGGAGTGGAACAGCCTCTGGATGACCCCCGTGGGGAACATGCTGTTCGACCAGGGCA
CCCTAGTGGGCGCCCTGGACTTCCGCAGCCTGCGGTCTCGGCACCCGTGGTCCGGGGAGCA
GGGGGCGTCGACCCGGGACGAGGGAAAACAATAA (SEQ ID NO: 4)
SEQ ID NO: 5 HSV-1 ICP0 amino acid sequence
Amino Acid Sequence for HSV-1 ICP0-strain KOS for e.g. variants. See e.g. also: Accession:
P08393.1 GI: 124134; Accession: AFI23590.1 GI: 384597746; Accession: AFI23649.1 GI: 384597805;
Accession: AFE62827.1 GI: 380776964; Accession: AFE62886.1 GI: 380777023; Accession:
ADM22381.1 GI: 304318198; Accession: ALO18731.1 GI: 952947655; Accession: ALO18672.1 GI:
952947596; Accession: ALO18655.1 GI: 952947578; Accession: ALO18596.1 GI: 952947519;
Accession: AKH80472.1 GI: 822581062; Accession: AKH80399.1 GI: 822580988; Accession:
AKG61929.1 GI: 820021112; Accession: AKG61857.1 GI: 820021035; etc. and the like.
MEPRPGASTRRPEGRPQREPAPDVWVFPCDRDLPDSSDSEAETE
VGGRGDADHHDDDSASEADSTDTELFETGLLGPQGVDGGAVSGGSPPREEDPGSCGGA
PPREDGGSDEGDVCAVCTDEIAPHLRCDTFPCMHRFCIPCMKTWMQLRNTCPLCNAKL
VYLIVGVTPSGSFSTIPIVNDPQTRMEAEEAVRAGTAVDFIWTGNQRFAPRYLTLGGH
TVRALSPTHPEPTTDEDDDDLDDADYVPPAPRRTPRAPPRRGAAAPPVTGGASHAAPQ
PAAARTAPPSAPIGPHGSSNTNTTTNSSGGGGSRQSRAAVPRGASGPSGGVGVVEAEA
GRPRGRTGPLVNRPAPLANNRDPIVISDSPPASPHRPPAAPMPGSAPRPGPPASAAAS
GPARPRAAVAPCVRAPPPGPGPRAPAPGAEPAARPADARRVPQSHSSLAQAANQEQSL
CRARATVARGSGGPGVEGGHGPSRGAAPSGAAPSGAPPLPSAASVEQEAAVRPRKRRG
SGQENPSPQSTRPPLAPAGAKRAATHPPSDSGPGGRGQGGPGTPLTSSAASASSSSAS
SSSAPTPAGATSSATGAASSSASASSGGAVGALGGRQEETSLGPRAASGPRGPRKCAR
KTRHAETSGAVPAGGLTRYLPISGVSSVVALSPYVNKTITGDCLPILDMETGNIGAYV
VLVDQTGNMATRLRAAVPGWSRRTLLPETAGNHVTPPEYPTAPASEWNSLWMTPVGNM
LFDQGTLVGALDFRSLRSRHPWSGEQGASTRDEGKQ (SEQ ID NO: 5)
SEQ ID NO: 6 HSV-2 ICP0 amino acid sequence ACCESSION YP_009137210
VERSION YP_009137210.1 GI:820945210 [note: for e.g. Variants, see also e.g. Accession:
YP_009137151.1 GI: 820945151; Accession: AEV91397.2 GI: 556197555; Accession: AEV91338.2 GI:
556197550;; Accession: ADG01890.1 GI: 295322885;; Accession: ADG01889.1 GI: 295322883;
Accession: ADG01888.1 GI: 295322881; Accession: ADG01887.1 GI: 295322879; Accession:
ADG01885.1 GI: 295322875; Accession: ADG01886.1 GI: 295322877; etc, and the like]]

| | |
|---|---|
| 1 | meprpgtssr adpgperppr qtpgtpaaph awgmlndmqw lassdseeet evgisdddlh |
| 61 | rdstseagst dtemfeaglm daatpparpp aerqgsptpa daqgscgggp vgeeeaeagg |
| 121 | ggdvcavctd eiapplrcqs fpclhpfcip cmktwiplrn tcplcntpva ylivgvtasg |
| 181 | sfstipivnd prtrveaeaa vragtavdfi wtgnqrtapr slslgghtvr alsptppwpg |
| 241 | tddedddlad vdyvppaprr aprrggggag atrgtsqpaa trpappgapr ssssggaplr |
| 301 | agvgsgsggg pavaavvprv aslppaaggg raqarrvged aaaaegrtpp agqpraaqep |
| 361 | pivisdsppp sprrpagpgp lsffssssaq vssgpggggl pqssgraarp raavaprvrs |
| 421 | ppraaaapvv sasadaagpa ppavpvdahr aprsrmtqaq tdtqaqslgr agatdargsg |
| 481 | gpgaeggpgv prgtntpgaa phaaegaaar prkrrgsdsg paasssasss aaprsplapq |
| 541 | gvgakraapr rapdsdsgdr ghgplapasa gaappsasps sqaavaaaas ssssaassss |
| 601 | saasssssaa sssssaasss saasssssss sasssaggag gsvasasgag erretslgpr |
| 661 | aaaprgprkc arktrhaegg pepgardpap gltrylpiag vssvvalapy vnktvtgdcl |
| 721 | pvldmetghi gayvvlvdqt gnvadllraa apawsrrtll peharncvrp pdyptppase |
| 781 | wnslwmtpvg nmlfdqgtlv galdfhglrs rhpwsreqga papagdapag hge |

SEQ ID NO: 7 Optimized tetR transcription unit: modified HCMV major immediate-early
promoter with VP16 responsive elements, beta-globin Intron, codon optimized tetR coding
sequence, and SV40 poly A signal sequence.
5′-
GAATTCACGCGTCCGTGCATGCTAATGATATTCCGCCCAACGACCCCCGCCCATTGACGTCA
ATAATGACGTATGTTCCCATAGTAACGCCAATAGGGACTTTCCATTGACGTCAATGGGTGGA
CTATTTACGGTAAACTGCATGCTAATGATATTCTTTGACTCACGGGGATTTCCAAGTCTCCAC
CCCATTGACGTCAATGGGAGTTTGTTTTGGCACCAAAATCAACGGGACTTTCCAAAATGTCG
TAACAACTCCGCCCCATTGACGCAAATGGGCGGTAGGCGTGTACGGTGGGAGGTCTATATA
AGCAGAGCTCGTTTAGTGAACCGTCAGATCGCCTGGAGACGCCATCCACGCTGTTTTGACCT
CCATAGAAGACACCGGGACCGATCCAGCCTCCGTCGCGAGGTGAGTTTGGGGACCCTTGAT
TGTTCTTTCTTTTTCGCTATTGTAAAATTCATGTTATATGGAGGGGGCAAAGTTTTCAGGGTG
TTGTTTAGAATGGGAAGATGTCCCTTGTATCACCATGGACCCTCATGATAATTTTGTTTCTTT
CACTTTCTACTCTGTTGACAACCATTGTCTCCTCTTATTTTCTTTTCATTTTCTGTAACTTTTTC
GTTAAACTTTAGCTTGCATTTGTAACGAATTTTTAAATTCACTTTTGTTTATTTGTCAGATTGT
AAGTACTTTCTCTAATCACTTTTTTTTCAAGGCAATCAGGGTATATTATATTGTACTTCAGCA
CAGTTTTAGAGAACAATTGTTATAATTAAATGATAAGGTAGAATATTTCTGCATATAAATTC
TGGCTGGCGTGGAAATATTCTTATTGGTAGAAACAACTACATCCTGGTCATCATCCTGCCTT
TCTCTTTATGGTTACAATGATATACACTGTTTGAGATGAGGATAAAATACTCTGAGTCCAAA
CCGGGCCCCTCTGCTAACCATGTTCATGCCTTCTTCTTTTTCCTACAGCTGGATCCGTGTTCC
AACCACGGTCACGCTTCGGTGGCCACCATGAGCAGACTGGACAAGAGCAAGGTGATCAACA
GCGCCCTGGAGCTGCTGAACGAGGTGGGCATCGAGGGCCTGACCACCAGAAAGCTGGCCCA
GAAGCTGGGCGTGGAGCAGCCCACCCTGTACTGGCACGTGAAGAACAAGAGAGCCCTGCTG
GACGCCCTGGCCATCGAGATGCTGGACAGACACCACACCCACTTCTGCCCCCTGGAGGGCG
AGAGCTGGCAGGACTTCCTGAGAAACAACGCCAAGAGCTTCAGATGCGCCCTGCTGAGCCA
CAGAGACGGCGCCAAGGTGCACCTGGGCACCAGACCCACCGAGAAGCAGTACGAGACCCT
GGAGAACCAGCTGGCCTTCCTGTGCCAGCAGGGCTTCAGCCTGGAGAACGCCCTGTACGCC
CTGAGCGCCGTGGGCCACTTCACCCTGGGCTGCGTGCTGGAGGACCAGGAGCACCAGGTGG
CCAAGGAGGAGAGAGAGACCCCCACCACCGACAGCATGCCCCCCCTGCTGAGACAGGCCAT
CGAGCTGTTCGACCACCAGGGCGCCGAGCCCGCCTTCCTGTTCGGCCTGGAGCTGATCATCT
GCGGCCTGGAGAAGCAGCTGAAGTGCGAGAGCGGCAGCTAAATAGGTAGGTAGTCGACCC
GGGACGAGGGAAAACAATAACAGAACTTGTTTATTGCAGCTTATAATGGTTACAAATAAAG
CAATAGCATCACAAATTTCACAAATAAAGCATTTTTTTCACTGCATTCTAGTTGTGGTTTGTC
CAAACTCATCAATGTATCTTATCATGTCTGAAGCTTCTGCAG-3′ (SEQ ID NO: 7)
SEQ ID NO: 8 Codon optimized tetR DNA coding sequence plus Kozak consensus sequence
GCCACCATGAGCAGACTGGACAAGAGCAAGGTGATCAACAGCGCCCTGGAGCTGCTGAACG
AGGTGGGCATCGAGGGCCTGACCACCAGAAAGCTGGCCCAGAAGCTGGGCGTGGAGCAGC
CCACCCTGTACTGGCACGTGAAGAACAAGAGAGCCCTGCTGGACGCCCTGGCCATCGAGAT
GCTGGACAGACACCACACCCACTTCTGCCCCCTGGAGGGCGAGAGCTGGCAGGACTTCCTG
AGAAACAACGCCAAGAGCTTCAGATGCGCCCTGCTGAGCCACAGAGACGGCGCCAAGGTGC
ACCTGGGCACCAGACCCACCGAGAAGCAGTACGAGACCCTGGAGAACCAGCTGGCCTTCCT
GTGCCAGCAGGGCTTCAGCCTGGAGAACGCCCTGTACGCCCTGAGCGCCGTGGGCCACTTC
ACCCTGGGCTGCGTGCTGGAGGACCAGGAGCACCAGGTGGCCAAGGAGGAGAGAGAGACC
CCCACCACCGACAGCATGCCCCCCCTGCTGAGACAGGCCATCGAGCTGTTCGACCACCAGG
GCGCCGAGCCCGCCTTCCTGTTCGGCCTGGAGCTGATCATCTGCGGCCTGGAGAAGCAGCTG
AAGTGCGAGAGCGGCAGCTAA (SEQ ID NO: 8)
SEQ ID NO: 9 Modified HCMV major immediate-early promoter, which has HCMV promoter
sequence from -174 to -370 bp deleted and contains two HSV-1 VP16 responsive elements
the first VP16 responsive element at position 149 bp and the second VP16 element at
position 274 bp upstream of the HCMV TATA element:
5′-
ATGCTAATGATATTCCGCCCAACGACCCCCGCCCATTGACGTCAATAATGACGTATGTTCCC
ATAGTAACGCCAATAGGGACTTTCCATTGACGTCAATGGGTGGACTATTTACGGTAAACTGC
ATGCTAATGATATTCTTTGACTCACGGGGATTTCCAAGTCTCCACCCCATTGACGTCAATGG
GAGTTTGTTTTGGCACCAAAATCAACGGGACTTTCCAAAATGTCGTAACAACTCCGCCCCAT
TGACGCAAATGGGCGGTAGGCGTGTACGGTGGGAGGTCTATATAAGCAGAGCTCGTTTAGT
GAACCGTCAGATCGCCTGGAGACGCCATCCACGCTGTTTTGACCTCCATAGAAGACAC
CGGGACCGAT CCAGCCTCCG (SEQ ID NO: 9)
Underlined-_VP16 responsive
SEQ ID NO: 10 Beta-globin intron
5′-
AGGTGAGTTTGGGGACCCTTGATTGTTCTTTCTTTTTCGCTATTGTAAAATTCATGTTATATG
GAGGGGGCAAAGTTTTCAGGGTGTTGTTTAGAATGGGAAGATGTCCCTTGTATCACCATGGA
CCCTCATGATAATTTTGTTTCTTTCACTTTCTACTCTGTTGACAACCATTGTCTCCTCTTATTT
TCTTTTCATTTTCTGTAACTTTTTCGTTAAACTTTAGCTTGCATTTGTAACGAATTTTTAAATT
CACTTTTGTTTATTTGTCAGATTGTAAGTACTTTCTCTAATCACTTTTTTTTCAAGGCAATCAG
GGTATATTATATTGTACTTCAGCACAGTTTTAGAGAACAATTGTTATAATTAAATGATAAGG
TAGAATATTTCTGCATATAAATTCTGGCTGGCGTGGAAATATTCTTATTGGTAGAAACAACT
ACATCCTGGTCATCATCCTGCCTTTCTCTTTATGGTTACAATGATATACACTGTTTGAGATGA
GGATAAAATACTCTGAGTCCAAACCGGGCCCCTCTGCTAACCATGTTCATGCCTTCTTCTTTT
TCCTACAGCT-3′ (SEQ ID NO: 10)
SEQ ID NO: 11 Codon optimized tetR DNA coding sequence plus Kozak consensus sequence and
28 bp of ICP27 5′ UTR (underlined)
GTGTTCCA ACCACGGTCA CGCTTCGGTG GCCACC
ATGAGCAGACTGGACAAGAGCAAGGTGATCAACAGCGCCCTGGAGCTGCTGAACGAGGTG
GGCATCGAGGGCCTGACCACCAGAAAGCTGGCCCAGAAGCTGGGCGTGGAGCAGCCCACC
CTGTACTGGCACGTGAAGAACAAGAGAGCCCTGCTGGACGCCCTGGCCATCGAGATGCTGG
ACAGACACCACACCCACTTCTGCCCCCTGGAGGGCGAGAGCTGGCAGGACTTCCTGAGAAA
CAACGCCAAGAGCTTCAGATGCGCCCTGCTGAGCCACAGAGACGGCGCCAAGGTGCACCTG
GGCACCAGACCCACCGAGAAGCAGTACGAGACCCTGGAGAACCAGCTGGCCTTCCTGTGCC
AGCAGGGCTTCAGCCTGGAGAACGCCCTGTACGCCCTGAGCGCCGTGGGCCACTTCACCCT
GGGCTGCGTGCTGGAGGACCAGGAGCACCAGGTGGCCAAGGAGGAGAGAGAGACCCCCAC
CACCGACAGCATGCCCCCCCTGCTGAGACAGGCCATCGAGCTGTTCGACCACCAGGGCGCC
GAGCCCGCCTTCCTGTTCGGCCTGGAGCTGATCATCTGCGGCCTGGAGAAGCAGCTGAAGTG
CGAGAGCGGCAGCTAA ATAGGTAGGTA (SEQ ID NO: 11)

1.-27. (canceled)

28. A method of producing a recombinant protein of interest comprising propagating a Vero cell line for sufficient time to allow for expression of the protein,
wherein the Vero cell line comprises a nucleotide sequence that encodes for a Herpes Simplex virus-1 (HSV-1) ICP0 protein operably linked to a promoter having a sequence of SEQ ID NO: 2.

29. The method of claim 28, wherein the nucleotide sequence that encodes HSV-1 ICP0 encodes the amino acid sequence of SEQ ID NO: 5.

30. The method of claim 28, wherein the Vero cell line further comprises a nucleic acid encoding the recombinant protein of interest.

31. The method of claim 30, wherein the nucleic acid encoding the recombinant protein of interest that is operably linked to a promoter.

32. The method of claim 28, wherein the recombinant protein of interest is a therapeutic protein.

33. The method of claim 32, wherein the therapeutic protein is a vaccine protein.

34. The method of claim 28, wherein the cell line exhibits Vero cell morphology.

35. The method of claim 28, wherein the cell line is capable of maintaining ICP0 complementation efficiency within 2 standard deviations as measured by a viral replication assay for greater than 20 cell passages, 30 cell passages, or 40 cell passages.

36. The method of claim 28, wherein the Vero cell line further comprises an additional nucleic acid that encodes a tetracycline repressor protein operably linked to a promoter.

37. The method of claim 36, wherein the promoter operably linked to the tetracycline repressor protein comprises SEQ ID NO: 9.

38. The method of claim 36, wherein the nucleic acid that encodes a tetracycline repressor protein has the sequence of SEQ ID NO: 11.

39. The method of claim 36, wherein the nucleic acid that encodes tetracycline repressor protein operably linked to a promoter comprises or consists essentially of SEQ ID NO: 7.

40. The method of claim 28, wherein the Vero cell line further comprises a gene encoding antibiotic resistance.

41. The method of claim 28, wherein the Vero cell line is used for propagating and production of an ICP0 deficient HSV virus.


SEQUENCE LISTING (12 sequences)

SEQ ID NO: 1 - DNA, 50 bp, synthetic construct
  misc_feature 1..50: note = Description of Artificial Sequence: Synthetic oligonucleotide
  source 1..50: mol_type = other DNA
ccattgacgcaaatgggcggtaggcgtgtacggtgggaggtctatataag

SEQ ID NO: 2 - DNA, 151 bp, synthetic construct
  misc_feature 1..151: note = Description of Artificial Sequence: Synthetic polynucleotide
  source 1..151: mol_type = other DNA
atgctaatgatatacatgccacgtacttatggtgtctatgctaatgatattcgcaaatgggcggtagaccggtgaattcatgctaatgatattctttggtaccattgacgcaaatgggcggtaggcgtgtacggtgggaggtctatataag

SEQ ID NO: 3 - DNA, 3372 bp, synthetic construct
  misc_feature 1..3372: note = Description of Artificial Sequence: Synthetic polynucleotide
  source 1..3372: mol_type = other DNA
atggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcgggggggggagagggggaactcgtgggtgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggaggagggggggggagggaggaataggcgggcgggcgaggaaagggcgggccggggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtccgcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggcgccgccccctccggcgccgccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataacagaacttgtttattgcagcttataatggttacaaataaagcaatagcatcacaaatttcacaaataaagcatttttttcactgcattctagttgtggtttgtccaaactcatcaatgtatcttatcatgtctgaagcttggc

SEQ ID NO: 4 - DNA, 3513 bp, synthetic construct
  misc_feature 1..3513: note = Description of Artificial Sequence: Synthetic polynucleotide
  source 1..3513: mol_type = other DNA
atgctaatgatatacatgccacgtacttatggtgtctatgctaatgatattcgcaaatgggcggtagaccggtgaattcatgctaatgatattctttggtaccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacggactctagaggatccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcgggggggggagagggggaactcgtgggtgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggaggagggggggggagggaggaataggcgggcgggcgaggaaagggcgggccggggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtccgcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggcgccgccccctccggcgccgccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataa

SEQ ID NO: 5 - protein, 776 aa, Human alphaherpesvirus 1
  source 1..776: note = Herpes simplex virus 1
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAVPRGASGPSGGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPSGAPPLPSAASVEQEAAVRPRKRRGSGQENPSPQSTRPPLAPAGAKRAATHPPSDSGPGGRGQGGPGTPLTSSAASASSSSASSSSAPTPAGATSSATGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVTPPEYPTAPASEWNSLWMTPVGNMLFDQGTLVGALDFRSLRSRHPWSGEQGASTRDEGKQ

SEQ ID NO: 6 - protein, 833 aa, Human alphaherpesvirus 2
  source 1..833: note = Herpes simplex virus 2
MEPRPGTSSRADPGPERPPRQTPGTPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRAGTAVDFIWTGNQRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPAGQPRAAQEPPIVISDSPPPSPRRPAGPGPLSFFSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAAASSSSSAASSSSSAASSSSSAASSSSSAASSSSAASSSSSSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 7 - DNA, 1836 bp, synthetic construct
  misc_feature 1..1836: note = Description of Artificial Sequence: Synthetic polynucleotide
  source 1..1836: mol_type = other DNA
gaattcacgcgtccgtgcatgctaatgatattccgcccaacgacccccgcccattgacgtcaataatgacgtatgttcccatagtaacgccaatagggactttccattgacgtcaatgggtggactatttacggtaaactgcatgctaatgatattctttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgtcgcgaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctggatccgtgttccaaccacggtcacgcttcggtggccaccatgagcagactggacaagagcaaggtgatcaacagcgccctggagctgctgaacgaggtgggcatcgagggcctgaccaccagaaagctggcccagaagctgggcgtggagcagcccaccctgtactggcacgtgaagaacaagagagccctgctggacgccctggccatcgagatgctggacagacaccacacccacttctgccccctggagggcgagagctggcaggacttcctgagaaacaacgccaagagcttcagatgcgccctgctgagccacagagacggcgccaaggtgcacctgggcaccagacccaccgagaagcagtacgagaccctggagaaccagctggccttcctgtgccagcagggcttcagcctggagaacgccctgtacgccctgagcgccgtgggccacttcaccctgggctgcgtgctggaggaccaggagcaccaggtggccaaggaggagagagagacccccaccaccgacagcatgccccccctgctgagacaggccatcgagctgttcgaccaccagggcgccgagcccgccttcctgttcggcctggagctgatcatctgcggcctggagaagcagctgaagtgcgagagcggcagctaaataggtaggtagtcgacccgggacgagggaaaacaataacagaacttgtttattgcagcttataatggttacaaataaagcaatagcatcacaaatttcacaaataaagcatttttttcactgcattctagttgtggtttgtccaaactcatcaatgtatcttatcatgtctgaagcttctgcag

SEQ ID NO: 8 - DNA, 630 bp, synthetic construct
  misc_feature 1..630: note = Description of Artificial Sequence: Synthetic polynucleotide
  source 1..630: mol_type = other DNA
gccaccatgagcagactggacaagagcaaggtgatcaacagcgccctggagctgctgaacgaggtgggcatcgagggcctgaccaccagaaagctggcccagaagctgggcgtggagcagcccaccctgtactggcacgtgaagaacaagagagccctgctggacgccctggccatcgagatgctggacagacaccacacccacttctgccccctggagggcgagagctggcaggacttcctgagaaacaacgccaagagcttcagatgcgccctgctgagccacagagacggcgccaaggtgcacctgggcaccagacccaccgagaagcagtacgagaccctggagaaccagctggccttcctgtgccagcagggcttcagcctggagaacgccctgtacgccctgagcgccgtgggccacttcaccctgggctgcgtgctggaggaccaggagcaccaggtggccaaggaggagagagagacccccaccaccgacagcatgccccccctgctgagacaggccatcgagctgttcgaccaccagggcgccgagcccgccttcctgttcggcctggagctgatcatctgcggcctggagaagcagctgaagtgcgagagcggcagctaa

SEQ ID NO: 9 - DNA, 387 bp, synthetic construct
  misc_feature 1..387: note = Description of Artificial Sequence: Synthetic polynucleotide
  source 1..387: mol_type = other DNA
atgctaatgatattccgcccaacgacccccgcccattgacgtcaataatgacgtatgttcccatagtaacgccaatagggactttccattgacgtcaatgggtggactatttacggtaaactgcatgctaatgatattctttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccg

SEQ ID NO: 10 - DNA, 577 bp, unidentified
  misc_feature 1..577: note = Description of Unknown: Beta-globin intron sequence
  source 1..577: mol_type = unassigned DNA
aggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagct

SEQ ID NO: 11 - DNA, 669 bp, synthetic construct
  misc_feature 1..669: note = Description of Artificial Sequence: Synthetic polynucleotide
  source 1..669: mol_type = other DNA
gtgttccaaccacggtcacgcttcggtggccaccatgagcagactggacaagagcaaggtgatcaacagcgccctggagctgctgaacgaggtgggcatcgagggcctgaccaccagaaagctggcccagaagctgggcgtggagcagcccaccctgtactggcacgtgaagaacaagagagccctgctggacgccctggccatcgagatgctggacagacaccacacccacttctgccccctggagggcgagagctggcaggacttcctgagaaacaacgccaagagcttcagatgcgccctgctgagccacagagacggcgccaaggtgcacctgggcaccagacccaccgagaagcagtacgagaccctggagaaccagctggccttcctgtgccagcagggcttcagcctggagaacgccctgtacgccctgagcgccgtgggccacttcaccctgggctgcgtgctggaggaccaggagcaccaggtggccaaggaggagagagagacccccaccaccgacagcatgccccccctgctgagacaggccatcgagctgttcgaccaccagggcgccgagcccgccttcctgttcggcctggagctgatcatctgcggcctggagaagcagctgaagtgcgagagcggcagctaaataggtaggta

SEQ ID NO: 12 - DNA, 11 bp, synthetic construct
  misc_feature 1..11: note = Description of Artificial Sequence: Synthetic oligonucleotide
  source 1..11: mol_type = other DNA
caaatgggcgg